(19) United States
(12) Patent Application Publication
(10) Pub. No.: US 2008/0177151 A1
(43) Pub. Date: Jul. 24, 2008

(54) SYSTEM AND METHOD FOR THE ORIENTATION OF SURGICAL DISPLAYS

(76) Inventor: Christopher Horvath, Irvine, CA (US)

Correspondence Address: Armando Pastrana, Jr.;Alcon Research, Ltd., R&D Counsel (TB4-8), 6201 South Freeway, Fort Worth, TX, 76134-2099, US

(21) Appl. No.: 11/626,031

(22) Filed: Jan. 23, 2007

(51) Int. Cl. A61B 5/00

(52) U.S. Cl. 600/300

(57) ABSTRACT

Embodiments of the present invention provide a system and method for arranging information displayed on a surgical display. Embodiments of the present invention can arrange information automatically or manually to orient information upright. Further embodiments of the present invention can arrange the placement of information displayed on the screen based on the orientation of the screen.

### TECHNICAL FIELD OF THE INVENTION

The present invention relates to surgical displays. More particularly, embodiments of the present invention relate to orienting surgical displays. Even more particularly, embodiments of the present invention relate to systems and methods for arranging information in a surgical display based on the orientation of the display.

### BACKGROUND

Surgical equipment containing a display is usually installed (setup or mounted) in fixed specific orientations. Different mounting options are not feasible since the display requires a certain orientation to be easily read. Therefore, there is a need for a system and method which provides more flexible mounting options.

### SUMMARY OF THE INVENTION

Embodiments of the present invention provide a system and method for arranging information in a surgical display such that the information will appear up-right regardless of the orientation of the surgical display. One embodiment of the present invention includes a surgical apparatus comprising a display to display surgical information and a controller coupled to the display. The controller is configured to arrange information to appear upright in the display based on an indication of an orientation of the display. The arrangement of information can change based on the orientation of the display, but, according to various embodiments of the present invention, can preserve the relative importance of various pieces of information and the proximity of various pieces of information to controls of the surgical apparatus.

Another embodiment of the present invention can include a method comprising, receiving an indication of an orientation of a surgical display, and displaying surgical information to appear upright to a user in the surgical display in an arrangement selected from a plurality of arrangements based on the orientation of the surgical display.

Yet another embodiment of the present invention can comprise a computer readable medium storing a set of computer instructions, said set of computer instructions comprising instructions executable by a processor to receive an indication of orientation of a display and display surgical information to appear upright to a user in the display in an arrangement selected from a plurality of arrangements based on the orientation of the display.

Embodiments of the present invention provide the advantage that information displayed on a screen integrated into a surgical display can be oriented to appear up-right to a user regardless of the physical orientation of the screen on which the information is displayed. Information arranged to appear up-right to a user may be intuitively easier for the user to read or analyze. Embodiments of the present invention provide additional advantages in that a surgical display may be oriented in any manner of ways and the information displayed will appear up-right to a user.

### BRIEF DESCRIPTION OF THE FIGURES

A more complete understanding of the present invention and the advantages thereof may be acquired by referring to the following description, taken in conjunction with the accompanying drawings in which like reference numbers indicate like features and wherein:

FIG. 1 is a diagrammatic representation of one embodiment of a surgical display oriented so as to be up-right;

FIG. 2 is a diagrammatic representation of one embodiment of a surgical display oriented so as to be side-ways;

FIGS. 3a-3c are diagrammatic representations of embodiments of screens upon which information is arranged as to appear up-right.

FIG. 4 is a flow chart representing the steps of one embodiment of a method for arranging information in a surgical display; and

FIG. 5 is a block diagram of one embodiment of a controller for a screen for a surgical display in accordance with the present invention.

### DETAILED DESCRIPTION

Preferred embodiments of the invention are illustrated in the FIGURES, like numerals being used to refer to like and corresponding parts of the various drawings.

Surgical displays can be used to display information including data, images or other information to a user. Surgical displays can be mounted in a variety of orientations, e.g. up-right, upside-down, side-ways, etc. Embodiments of the present invention help ensure that the orientation of information displayed on a screen of a surgical display will appear up-right to a user viewing the surgical display.

Embodiments of the present invention enhance the flexibility of surgical displays by helping to ensure that information displayed on a screen of a surgical display will appear up-right to a user regardless of the physical orientation of the surgical display itself. Up-right information may be easier for a user to read, analyze or understand than information which does not appear up-right to the user. In addition, viewing information which appears up-right may be more comfortable for the user.

One embodiment of the present invention works as follows. In a surgical apparatus, an orientation sensor detects the orientation of the surgical display. The detected orientation is input to a controller which changes the arrangement of information in the display so that the information appears up-right to a user. In one embodiment of the invention, the orientation sensor is a gravity sensor.

Another embodiment of the present invention works as follows. In a surgical display apparatus, a user inputs an indication of an orientation of the display. The desired orientation is input to a controller which changes the arrangement of information displayed on a screen of the surgical display so that the information is oriented according to the user's desired orientation.

In either case, the arrangement of information displayed can change with the orientation of the display to ensure that information is displayed upright on the screen. Additionally, the arrangement can change to place information to correspond to the appropriate controls on the surgical display or surgical instrumentation. For example, data displayed on an up-right oriented screen may be located next to corresponding controls on the surgical display. In some embodiments of the invention, no matter the orientation of the surgical display, the information displayed on the screen will be arranged so that the data displayed on the screen will be located next to the corresponding controls on the surgical display. Furthermore, it may be desirable to maintain the hierarchy of importance of the information displayed. This can be achieved by, for example, maintaining the relative size of various pieces of information or otherwise arranging information.

In some embodiments of the invention, a screen integrated into the surgical display may be a touch screen operable to receive user input. The use of a touch screen may reduce or eliminate the need for other user input devices on the surgical display. The use of a touch screen may also eliminate the need to arrange information to correspond to corresponding controls on the surgical display when arranging information to appear upright.

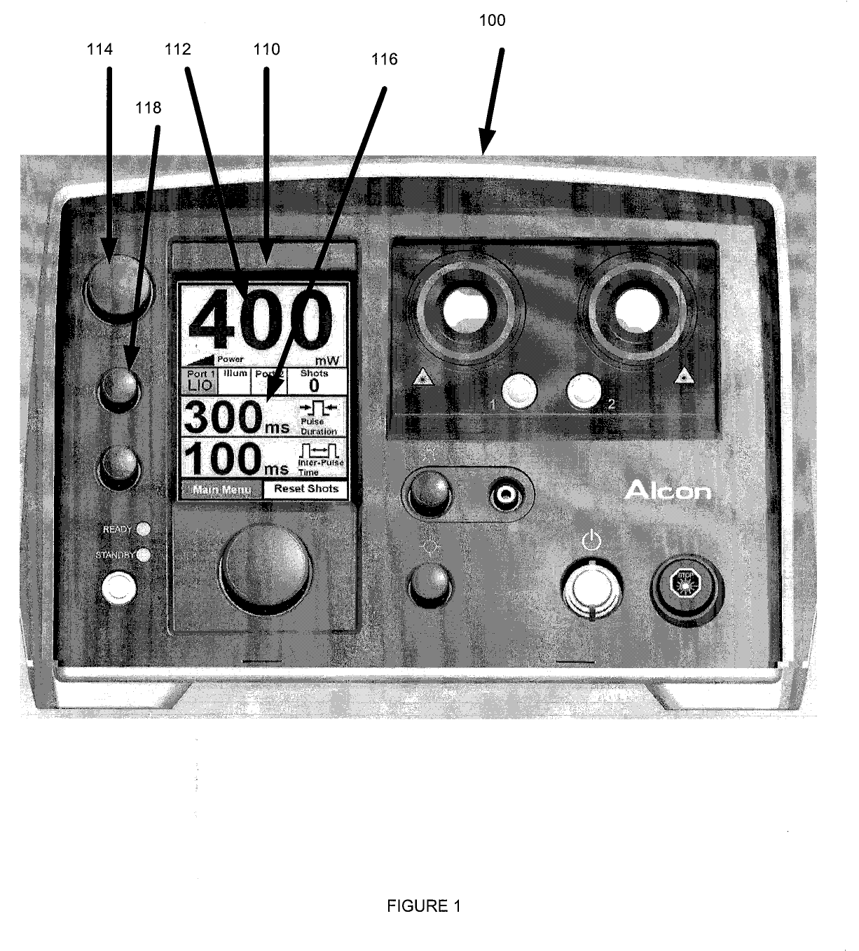

FIG. 1 is a diagrammatic representation of one embodiment of surgical instrumentation 100 (in this case a laser console manufactured by Alcon Laboratories, Inc. headquartered in Fort Worth, Tex.) having a surgical display 110 for surgical information. Surgical display 110 can comprise a touch screen, video screen or other display known or developed in the art. Display 110 can display information (including data, images and other information). In operation, information displayed in display 110 can be arranged to appear up-right regardless of the orientation of surgical instrumentation 100. Additionally, the information can be further arranged so that particular pieces of information in the display are located proximate to particular controls. For example, the information in portion 112 can always be displayed near control knob 114 and the information in portion 116 can always be displayed near control knob 118.

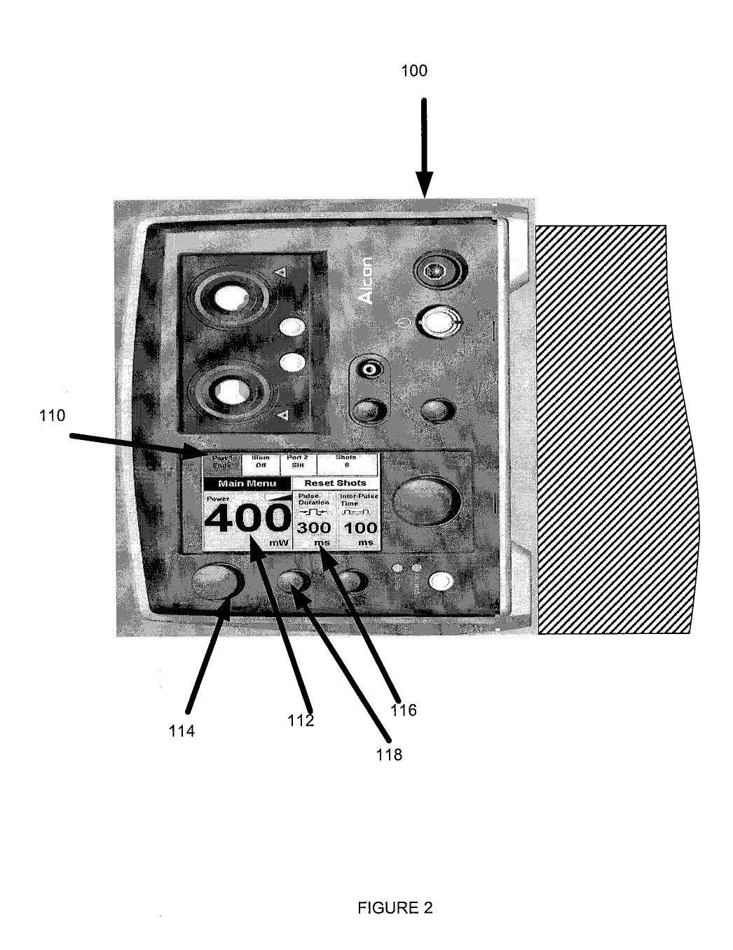

FIG. 2 is a diagrammatic representation of surgical instrumentation 100 mounted in a sideways configuration. In accordance with embodiments of the invention, the information in display 110 has been arranged so that the information will appear up-right to a user. In this embodiment of the invention, the information in display 110 is arranged differently than the information in FIG. 1 so that the information in portion 112 is still displayed near control knob 114 and the information in section 116 is displayed near control knob 118 and so on. However, the size of various pieces of information in display 110 relative to each other can be and has been maintained, thus preserving a visual hierarchy of importance among the various pieces of information.

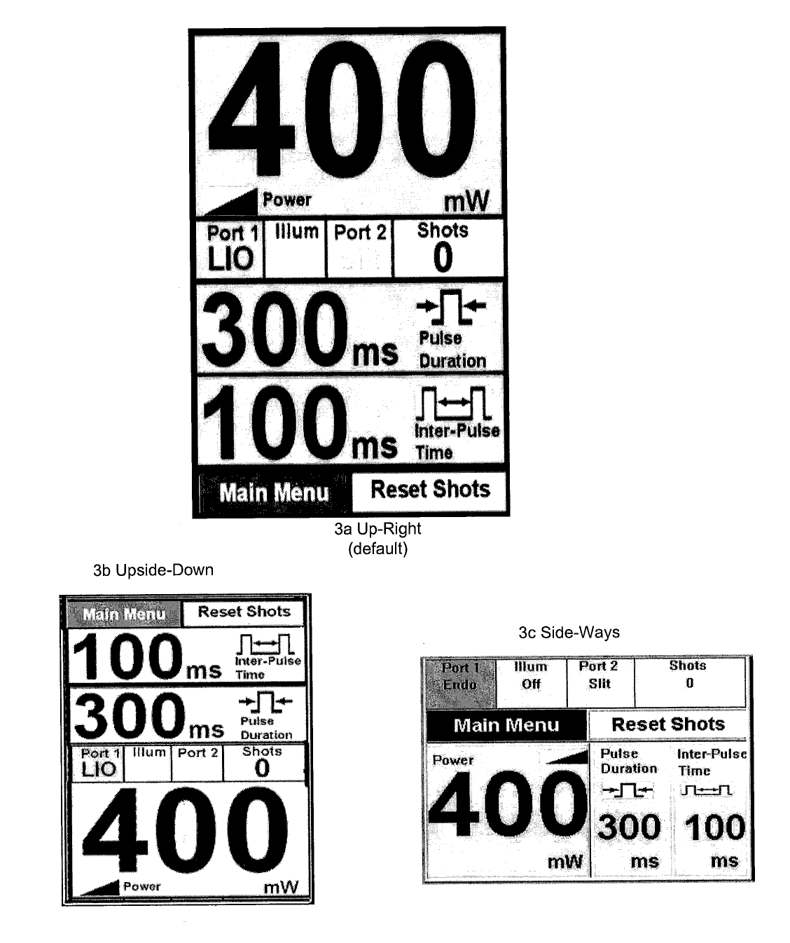

FIGS. 3a-3c are diagrammatic representations of embodiments of various arrangements of information so as to appear up-right. FIG. 3a is one embodiment of information displayed on an up-right oriented screen. In this embodiment of the invention, this arrangement is the default display and arrangement of information. FIG. 3b is one embodiment of information displayed on an upside-down oriented screen. In FIG. 3b, the arrangement of information differs from the arrangement of information shown in FIG. 3a. However, the relative size of the various pieces of information has been preserved. Additionally, when compared to FIG. 1, the relative placement of particular pieces of information relative to various control knobs has also been preserved. FIG. 3c is one embodiment of information displayed on a side-ways oriented screen. Again, while the arrangement of information differs from that shown in FIG. 3a, the relative size of information (or other indication of importance) has remained and the positioning of particular pieces of information near particular controls has also been maintained.

The arrangements of FIGS. 3a-3c are by way of example only and are not to be construed in a limiting sense: other arrangements are possible.

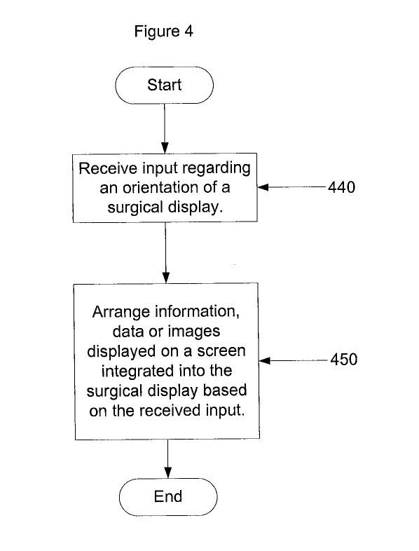

FIG. 4 is a flow chart representing the steps of one embodiment of a method of the present invention for orienting information on a screen used to display surgical information. The method of FIG. 4 can be facilitated through a set of computer executable instructions stored on a computer readable medium at, for example, the surgical instrumentation. At step 440, input is received regarding the orientation of a surgical display. This input could be based on, for example, data from an orientation sensor such as a gravity sensor or user input from a user input device such a control or touch screen input. The sensor may be capable of generating outputs for two or more orientations, but preferably can indicate four orientations (right side-up, upside-down, on one side, on the other side). At step 450, information displayed on the screen is arranged to appear up-right to a user based on the input received regarding the orientation of the surgical display. Additionally, the information can be arranged to preserve an indication of importance and maintain the proximity of particular pieces of information to various controls in the screen or off the screen.

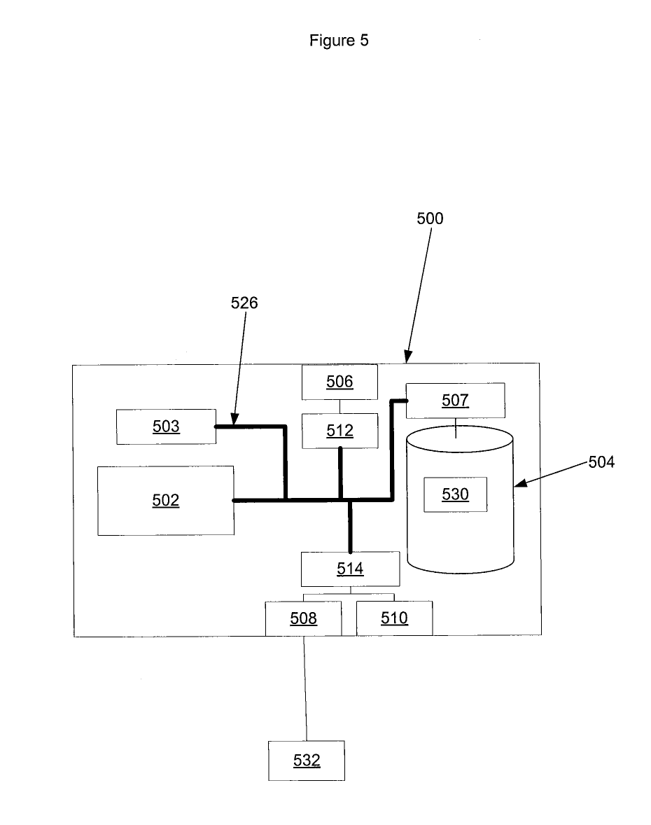

FIG. 5 is a diagrammatic representation of a surgical display controller 500 (“controller 500”). Controller 500 can include a processor 502, such as an Intel Pentium 4 based processor (Intel and Pentium are trademarks of Intel Corporation of Santa Clara, Calif.), a primary memory 503 (e.g., RAM, ROM, flash memory, EEPROM or other computer readable medium known in the art) and a secondary memory 504 (e.g., a hard drive, disk drive, optical drive or other computer readable medium known in the art). A memory controller 507 can control access to secondary memory 504. Controller 500 can include I/O interfaces, such as display interface 506. A video controller 512 can control interactions over display interface 506. Similarly, an I/O controller 514 can control interactions over I/O interfaces 508 and 510. Controller 500 can include a variety of input devices. Various components of controller 500 can be connected by a bus 526.

Secondary memory 504 can store a variety of computer instructions that include, for example, an operating system such as a Windows operating system (Windows is a trademark of Redmond, Wash. based Microsoft Corporation) and applications that run on the operating system, along with a variety of data. More particularly, secondary memory 504 can store a software program 530 that orients information, data and images displayed on a screen of the surgical display based on input, such as orientation input based on data from an orientation sensor. During execution by processor 502, portions of program 530 can be stored in secondary memory 504 and/or primary memory 503.

In operation, program 530 can be executable by processor 502 to arrange information based on input. This input can come from an orientation sensor such as orientation sensor 532 (e.g., a gravity sensor or other orientation sensor known or developed in the art) or from a user input device such as a control integrated into the surgical display. Thus, the controller 500 can arrange information such that the information is up-right to a user.

Controller 500 of FIG. 5 is provided by way of example only and it should be understood that embodiments of the present invention can be implemented as a set of computer instructions stored on a computer readable medium in a variety of computing devices. Program 530 can be executable to receive and store data over a network and can include instructions that are stored at a number of different locations and are executed in a distributed manner. While shown as a stand alone program in FIG. 5, it should be noted that program 530 can be a module of a larger program, can comprise separate programs operable to communicate data to each other or can be implemented according to any suitable programming architecture and language.

One advantage of arranging information based on an orientation of a display is that the surgical display itself can be oriented in a way that is convenient for a user. For example, a user may orient the surgical display upside-down so as to be able to operate controls on the surgical display without obstructing his view of the screen and the information will still appear up-right to the user. The surgical display may be mounted side-ways to conserve space or to comport with spatial restrictions and the information, data or images displayed on a screen of the surgical display will appear up-right to the user.

Embodiments of the present invention can be used in conjunction with a variety of medical systems, and can provide increased flexibility and ease of use. Users are able to place or mount a surgical display at a convenient orientation or an orientation which conserves space and the information displayed on a screen of the surgical display will be oriented such that they appear up-right to a user. In some embodiments, the information on the screen can be oriented based on both manually and automatically generated inputs, allowing enhanced flexibility.

Although the present invention has been described in detail herein with reference to the illustrated embodiments, it should be understood that the description is by way of example only and is not to be construed in a limiting sense. It is to be further understood, therefore, that numerous changes in the details of the embodiment of this invention and additional embodiments of this invention will be apparent, and may be made by, persons of ordinary skill in the art having reference to this description. It is contemplated that all such changes and additional embodiments are within scope of the invention as claimed below.

What is claimed is:

1. A surgical apparatus comprising:
a display to display surgical information; and
a controller coupled to the display, the controller configured to arrange information to appear upright in the display based on an indication of an orientation of the display.

2. The surgical apparatus of claim 1, further comprising an orientation sensor coupled to the controller, wherein the orientation sensor detects the orientation of the display and wherein the controller arranges information in the display based on the detected orientation.

3. The surgical apparatus of claim 2, wherein the orientation sensor is a gravity sensor.

4. The surgical apparatus of claim 1, further comprising a user input device, wherein the controller arranges information in the display based on user input from the user input device indicating the orientation of the display.

5. The surgical apparatus of claim 1, wherein the controller is configured to maintain a relative importance of various pieces of information in a plurality of arrangements.

6. The surgical display apparatus of claim 4, wherein the controller is further configured to:
arrange information in the display so that a first a piece of information is proximate to a first control in a first arrangement and a second piece of information is proximate to a second control in the first arrangement;
arrange information the display so that the first piece of information is proximate to the first control in the first arrangement and the second piece of information is proximate to the second control in the second arrangement; and
wherein the first arrangement corresponds to a first orientation of the display and the second arrangement corresponds to a second orientation of the display.

7. The surgical apparatus of claim 1, wherein the display comprises a touch screen.

8. A computer program product comprising a computer readable medium storing a set of computer instructions, said set of computer instructions comprising instructions executable by a processor to:
receive an indication of orientation of a display;
display surgical information to appear upright to a user in the display in an arrangement from a plurality of arrangements based on the orientation of the display.

9. The computer program product of claim 8, further comprising instructions to receive the indication of orientation from an orientation sensor.

10. The computer program product of claim 8, further comprising instructions to receive the indication of orientation from a user input device.

11. The computer program product of claim 8, wherein the set of instructions further comprise instructions executable to maintain a relative importance of various pieces of information in the plurality of arrangements.

12. The computer program product of claim 8, wherein the set of instructions further comprise instructions executable to arrange information in the display so that a first a piece of information is proximate to a first control in a first arrangement corresponding to a first orientation of the display and a second arrangement corresponding to a second orientation of the display and to arrange the information so that a second piece of information is proximate to a second control in the first arrangement and the second arrangement.

13. A method comprising:
receiving an indication of an orientation of a surgical display;
displaying surgical information to appear upright to a user in the surgical display in an arrangement from a plurality of arrangements based on the orientation of the surgical display.

14. The method of claim 13, further comprising receiving the indication of the orientation of the surgical display from a user input device.

15. The method of claim 13, further comprising receiving the indication of the orientation from an orientation sensor.

16. The method of claim 13, further comprising maintaining a relative importance of various pieces of information in the plurality of arrangements.

17. The method of claim 13, further comprising arranging surgical information in the surgical display so that a first a piece of information is proximate to a first control in a first arrangement and a second piece of information is proximate to a second control in a second arrangement; and
arranging the surgical information in the surgical display so that the first piece of information is proximate to the first control in a second arrangement and the second piece of information is proximate to the second control in a second arrangement;
wherein the first and second arrangements correspond to different orientations of the surgical display.
